(19) United States
(12) Patent Application Publication
(10) Pub. No.: US 2019/0336306 A1
(43) Pub. Date: Nov. 7, 2019

(54) METHOD FOR REVISION ARTHROPLASTY

(71) Applicant: OLYMPUS CORPORATION, Tokyo (JP)

(72) Inventors: Manabu ISHIKAWA, Hachioji-shi (JP); Chie ONUMA, Tama-shi (JP)

(73) Assignee: OLYMPUS CORPORATION, Tokyo (JP)

(21) Appl. No.: 16/515,090

(22) Filed: Jul. 18, 2019

Related U.S. Application Data
(63) Continuation-in-part of application No. 15/085,521, filed on Mar. 30, 2016, now Pat. No. 10,368,895.
(60) Provisional application No. 62/268,931, filed on Dec. 17, 2015.

(51) Int. Cl. A61F 2/46; A61F 2/36; A61F 2/34

(52) U.S. Cl. CPC A61F 2/4607; A61F 2/3601; A61F 2002/3069; A61F 2/4609; A61F 2002/4683; A61F 2/34

(57) ABSTRACT

A method for revision arthroplasty of an embodiment is detaching an existing femur component of an artificial joint from a femur, the existing femur component being fixed by cement, and the artificial joint being composed of the existing femur component and an acetabulum component; breaking and removing the cement and bone remaining in an attaching of the femur from which the existing femur component was detached, by transmitting ultrasonic vibration to a treating section provided at a probe distal end of an ultrasonic treatment device when the treating section is brought into contact with the cement and the bone, and regenerating an attaching hole for a new femur component; applying cement in the regenerated attaching hole; and attaching and fixing a new femur component in the regenerated attaching hole.

### CROSS-REFERENCE TO RELATED APPLICATIONS

This application is a Continuation-in-Part Application of U.S. patent application Ser. No. 15/085,521, filed Mar. 30, 2016 and claiming the benefit of U.S. Provisional Application No. 62/268,931 filed Dec. 17, 2015, the entire contents of which are incorporated herein by reference.

### FIELD

The present invention relates to a method for revision arthroplasty to prepare an artificial joint by use of an ultrasonic treatment device.

### BACKGROUND

In general, an artificial joint wears away and loosens due to long use. When this looseness is generated, a replacement operation is required again. However, when a user is very old, the user might hesitate to undergo the operation again in consideration of rehabilitation and the like.

In a case where this artificial joint is, for example, an artificial hip joint, a stem is disposed in a femur. In this case, the femur is cut into a shape to receive the stem (a material of the stem is usually a metal material such as a titanium alloy) with a drill or the like, but in this technique, accuracy of a cut surface is not much high. In a case where the accuracy is low, a clearance is present between the stem and the bone when the stem is attached, and hence friction is generated together with the looseness. Due to this friction, wear debris of the material might be generated. A situation (called bone absorption) occurs in which the worn material is recognized as a foreign matter on a biological side to cause a biological reaction, thereby melting the bone around the artificial joint, and it is considered that this situation increases the clearance around the stem of the artificial joint.

To eliminate such a problem, there is provided a technique to dispose an artificial joint that is hard to loosen even when the artificial joint is used for a long period of time. Furthermore, the technique can contribute to decrease of occurrence of pain, because the artificial joint less loosens.

In addition, in the case where an artificial joint is already attached in a user's body, if germs existing in the user's body adhere to the artificial joint, the vicinity of the joint is infected with the germs and an infection disease causing inflammation occurs, the revision of the artificial joint is needed.

### SUMMARY

According to an embodiment of the present invention, there is provided a method for revision arthroplasty, comprising: detaching an existing femur component of an artificial joint from a femur, the existing femur component being fixed to the femur by cement, and the artificial joint being composed of the existing femur component and an acetabulum component; breaking and removing the cement and bone remaining in an attaching of the femur from which the existing femur component was detached, by transmitting ultrasonic vibration to a treating section provided at a probe distal end of an ultrasonic treatment device when the treating section is brought into contact with the cement and the bone, and regenerating an attaching hole of the femur for a new femur component; applying cement in the regenerated attaching hole of the femur; and attaching and fixing a new femur component in the regenerated attaching hole of the femur.

According to another embodiment of the present invention, there is provided a method for revision arthroplasty, comprising: detaching an existing acetabulum component of an artificial joint from an acetabulum, the existing acetabulum component being fixed to the acetabulum by cement, and the artificial joint being composed of the existing acetabulum component and a femur component; breaking and removing the cement and bone remaining in the acetabulum from which the existing acetabulum component was detached, by transmitting ultrasonic vibration to a treating section provided at a probe distal end of an ultrasonic treatment device when the treating section is brought into contact with the cement and the bone, and regenerating the acetabulum for a new acetabulum component; applying cement to the regenerated acetabulum; and attaching and fixing the new acetabulum component in the regenerated acetabulum.

Advantages of the invention will be set forth in the description which follows, and in part will be obvious from the description, or may be learned by practice of the invention. The advantages of the invention may be realized and obtained by means of the instrumentalities and combinations particularly pointed out hereinafter.

### BRIEF DESCRIPTION OF THE DRAWINGS

The accompanying drawings, which are incorporated in and constitute a part of the specification, illustrate embodiments of the invention, and together with the general description given above and the detailed description of the embodiments given below, serve to explain the principles of the invention.

FIG. 1 is a diagram showing a constitution example of a surgical system comprising an ultrasonic treatment device to carry out replacement arthroplasty of the present embodiment;

FIG. 2 is a view showing a constitution example of the ultrasonic treatment device in the present surgical system;

FIG. 3A is a view showing positional information of an attaching surface to attach, to a femur, a U-shaped implant that becomes an artificial knee joint;

FIG. 3B is a view showing a state of attaching the implant to the attaching surface of the femur;

FIG. 3C is a view showing a state where the implant is attached to the femur;

FIG. 4A is a view showing a state of cutting a tip of a tibia;

FIG. 4B is a view showing a state of forming a tip of the tibia in a bonding surface of a base plate;

FIG. 4C is a view of an assembly of the base plate and an articular facet surface which are to be attached to the tibia;

FIG. 5 is a flowchart to explain surgical steps of a method for replacement arthroplasty of an artificial hip joint;

FIG. 6 is a view showing a pelvis and a femur which become targets of a replacement operation;

FIG. 7A is a view to explain severance of the femur;

FIG. 7B is a view showing a state of a flattening treatment of the attaching surface;

FIG. 7C is a view showing the flattened attaching surface;

FIG. 7D is a view showing a state of forming a medullary cavity of the femur in a stem attaching hole by use of an ultrasonic treatment device;

FIG. 8 is a view showing appearances of a femur component and the femur in which the medullary cavity is formed;

FIG. 9A is a view showing an acetabulum of the pelvis that becomes a receiving port of the femur component;

FIG. 9B is a view of performing a smoothening treatment of the acetabulum by use of the ultrasonic treatment device;

FIG. 10 is a view showing a state where the femur component is attached to the pelvis;

FIG. 11 is a view to explain surgical steps of the method for the replacement arthroplasty of the artificial hip joint;

FIG. 12 is a view showing an artificial hip joint which is a target of a revision operation;

FIG. 13 is a view showing a state of a femur which is a target of the revision operation;

FIG. 14 is a view showing a state in which an attaching hole of a femur medullary cavity, from which a stem was removed, is treated by an ultrasonic treatment device;

FIG. 15A is a view showing a state of an acetabulum at a time when an acetabulum component was removed from the pelvis;

FIG. 15B is a view showing a state in which cement in the acetabulum and a remaining portion of the acetabulum component are removed by the ultrasonic treatment device, and a smoothing treatment is performed;

FIG. 16 is a flowchart to explain surgical steps of the revision of a femur component in the method for revision arthroplasty;

FIG. 17 is a flowchart to explain surgical steps of the revision of an acetabulum component in the method for revision arthroplasty; and

FIG. 18 is a flowchart to explain surgical steps of the method for revision arthroplasty.

### DETAILED DESCRIPTION

Hereinafter, with reference to the drawings, there will be described a method for replacement arthroplasty to prepare an artificial joint according to an embodiment of the present invention. In the following respective embodiments, there will be described two examples where treatment target regions are a knee joint and a hip joint, but the regions are not limited to these joints, and it is also possible to easily carry out a surgery for another joint by use of an ultrasonic treatment device in the same manner.

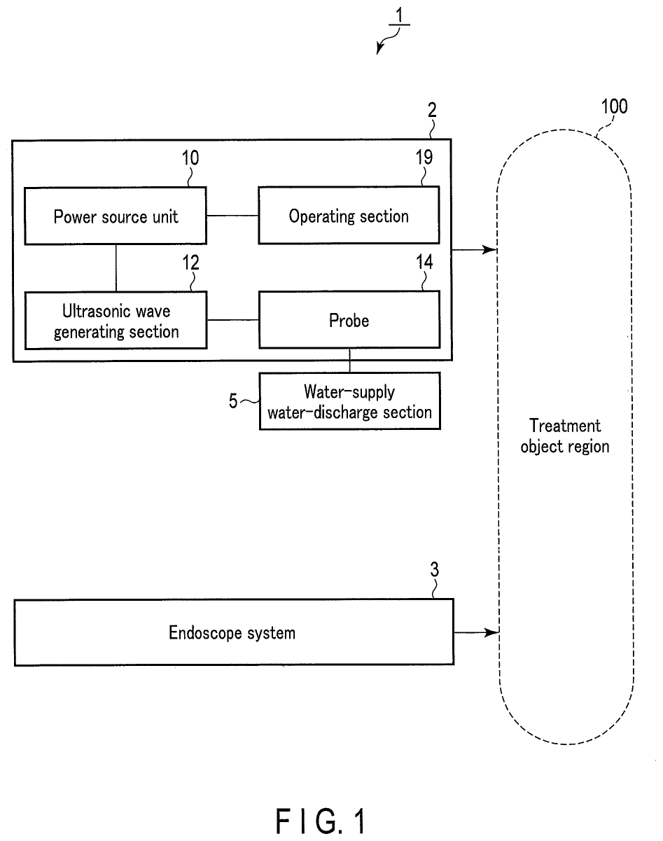

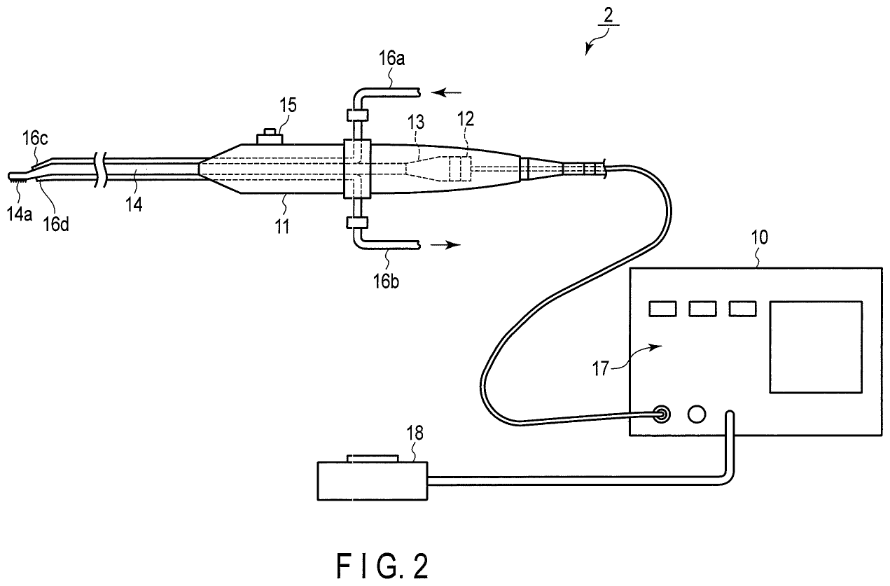

FIG. 1 shows a constitution example of a surgical system comprising the ultrasonic treatment device to carry out replacement arthroplasty of the present embodiment. FIG. 2 is a view showing a constitution example of the ultrasonic treatment device in the present surgical system.

A surgical system 1 of the present embodiment is constituted of an ultrasonic treatment device 2, an endoscope system 3 including an arthroscope, and a water-supply water-discharge section 5 that supplies and discharges the perfusion fluid such as saline.

The ultrasonic treatment device 2 comprises an ultrasonic wave generating section 12 that generates ultrasonic vibration by an ultrasonic vibration element (e.g., a piezoelectric element), a probe 14 that transmits the ultrasonic vibration to perform a cutting treatment of the treatment target region, a power source unit 10 that supplies a driving power to the ultrasonic wave generating section 12, and an operating section 19 to perform an on/off operation of the generation of the ultrasonic vibration. By the ultrasonic treatment device 2, a cutting treatment and an incising treatment are performed to treatment target regions such as a biological tissue, a cartilage and a bone (a subchondral bone) by use of the ultrasonic vibration.

The water-supply water-discharge section 5 supplies the perfusion fluid such as the saline to a periphery of a treatment object region 100 including a joint through the ultrasonic treatment device 2, discharges the perfusion fluid from the periphery, and thus circulates the perfusion fluid at a constant flow rate. In the present embodiment, the supplying and discharging are performed through the water-supply water-discharge section, but the supplying and discharging may be performed through the endoscope system 3.

Although not shown, the endoscope system 3 is constituted of the arthroscope made of a hard mirror that is one type of endoscope, a visible light source that is a light source of illumination light for irradiation with the illumination light of visible light, a control section that controls the whole endoscope system 3, an input section such as a keyboard or a touch panel, and a display section that displays surgical information including a photographed surgical situation.

The ultrasonic treatment device 2 of the present embodiment will be described in detail.

As shown in FIG. 2, the ultrasonic treatment device 2 of the present embodiment is constituted of a treatment device main body 11 comprising the ultrasonic wave generating section 12 and the probe 14, the power source unit 10, and a foot switch 18 that instructs the on/off operation of the generation of the ultrasonic vibration. The treatment device main body 11 is connected to the power source unit 10 via a cable, thereby performing the supply of the driving power and communication of a control signal.

The treatment device main body 11 comprises the ultrasonic wave generating section 12 having a tubular shape to be grasped by an operator and including therein an ultrasonic vibrator (a piezoelectric member or the like), the thin and long probe 14 having its proximal side acoustically connected to the ultrasonic wave generating section 12 via a horn 13, a treating section 14a disposed at a distal end of the probe to perform cutting, an operation switch 15 disposed on the treatment device main body 11 to instruct the on/off operation of the generation of the ultrasonic vibration, and flow channels 16a and 16b to supply and discharge the perfusion fluid from the water-supply water-discharge section 5 to circulate the perfusion fluid. The foot switch 11 has a function identical to that of the operation switch 15.

Although not shown, the treating section 14a is provided with projections having edges, the cartilage can be cut off by the edges, and the cartilage can be melted and cut off by friction heat generated between the treating section 14 and the cartilage. Additionally, bones (a cortical bone and a cancellous bone) such as the subchondral bones can be hammered with the above-mentioned projections of the treating section 14a by use of the ultrasonic vibration in the same manner as in a hammer (hammering), and the bone can remarkably finely be ground and cut off.

### First Embodiment

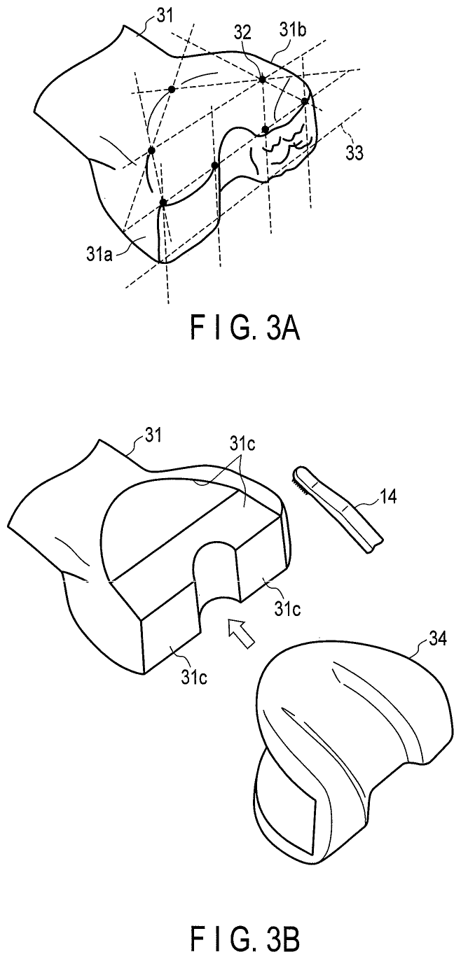

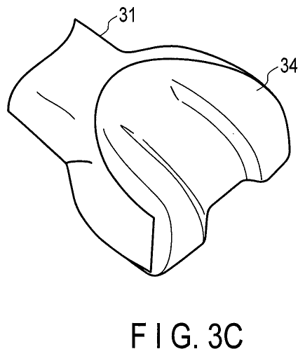

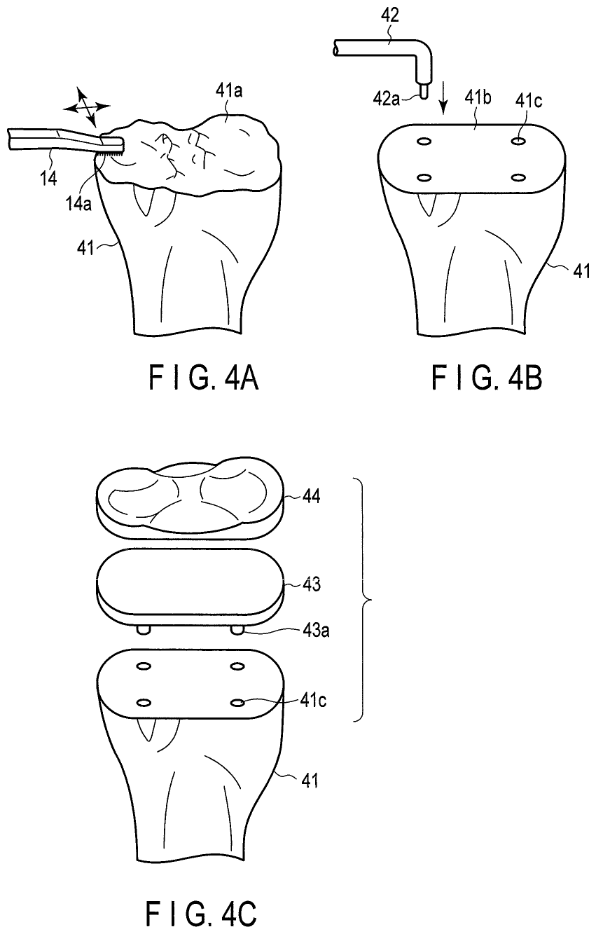

Next, with reference to FIG. 3A, FIG. 3B and FIG. 4A to FIG. 4D, there will be described a method for replacement arthroplasty of an artificial knee joint according to a first embodiment. FIG. 3A is a view showing positional information of an attaching surface to attach, to a femur, a U-shaped implant that becomes the artificial knee joint, FIG. 3B is a view showing a state of attaching the implant to the attaching surface of the femur, and FIG. 3C is a view showing a state where the implant is attached to the femur. FIG. 4A is a view showing a state of cutting a tip of a tibia, FIG. 4B is a view showing a state of forming the tip of the tibia in a bonding surface of a base plate, and FIG. 4C is a view of an assembly of the base plate and an articular facet surface which are to be attached to the tibia.

This artificial knee joint comprises two joint coupled regions, and is constituted of a femur implant 34 that becomes a first joint portion to be attached to a femur, and a base plate 43 and an articular facet surface 44 which constitute an attaching portion of a tibia implant and become a second joint portion to be attached to the tibia.

First, as shown in FIG. 3A, an attaching surface is positioned to tips 31a and 31 of a femur 31 to correspond to an attaching dimension of the femur implant to be attached (step S1). For this positioning setting, the positioning is performed by measuring an actual dimension of the femur 31 or actually bringing the femur implant into contact with the femur, and the femur is directly marked by using the ultrasonic treatment device 2. Furthermore, an image of the imaged femur 31 and information such as the attaching dimension of the femur implant 34 may be input into a personal computer to set a position of the attaching surface of the femur 31 on the image by image processing, and the femur may directly be marked by using the ultrasonic treatment device 2.

Next, as shown in FIG. 3B, under an environment (situation) in which the perfusion fluid circulates, the tip of the femur is cut and formed to match an attaching surface of the femur implant 34, in accordance with the above-mentioned positioning setting by use of the ultrasonic treatment device 2 (step S2). At this time, the femur 31 is roughly cut into an approximate size that is larger than the attaching dimension of the femur implant 34, and then the femur may finely be adjusted by bringing the femur implant 34 into contact with the femur.

Furthermore, each cut surface 31c of the femur 31 and the attaching surface of the femur implant are flattened and finished to eliminate unevenness from the surfaces by use of the ultrasonic treatment device 2 (step S3). Through this flattening, the surfaces come in contact closely with each other and are fixed with increased strength at a time of attaching. In such an excising treatment of the tip of the femur, the tip of the femur can smoothly be excised with small force by use of the ultrasonic vibration of the ultrasonic treatment device, so that handling can easily be performed and the tip can accurately be excised along an intended line.

Next, as shown in FIG. 3C, the femur implant 34 is attached to the attaching surfaces 31c of the femur 31 and fixedly bonded thereto by use of an adhesive (step S4). The femur implant 34 may have a screwing fixing structure in which screws or the like are used.

Next, as shown in FIG. 4A, a tip of a tibia 41 is flatly cut by using the ultrasonic treatment device 2 under the environment in which the perfusion fluid circulates (step S5). Furthermore, a reaming treatment of a cutting and shaping treatment and a rasping treatment of a polishing treatment are successively performed, and an attaching surface 41b that becomes a receiving region of the base plate 43 is formed in the tibia 41 to match a shape of the bonding surface of the base plate made of a metal (step S6).

On the bonding surface (a back surface side) of the base plate 43 of the attaching portion of the tibia implant applied to the present embodiment, anchors 43a comprising four cylindrical projections are disposed. As shown in FIG. 4B, cylindrical concave regions 41c to fit with the anchors 43a are formed in the attaching surface 41b of the tibia 41 (step S7). The concave regions 41c are formed by using the ultrasonic treatment device 2 in which an L-shaped probe 42 comprises a treating section 42a having a diameter equal to or smaller than that of the anchor 43a and including a pointed end portion that is bent in its middle and has a pointed distal end. The treating section 42a is pressed against the surface in a direction intersecting a longitudinal direction of the probe 42 to make a hole by cutting the surface. Therefore, the treating section is suitable for a case where a space to perform the cutting is narrow, or the like.

The probe 42 has the shape bent at a right angle, but a bending angle is not limited to the right angle and may be any angle. Additionally, the probe does not have to be necessarily bent, and may comprise the treating section 42a extending in the same direction as the longitudinal direction of the probe 42. In this case, the probe 42 is held vertically to the attaching surface 41b of the tibia 41 and pressed against the surface in a vertical direction to make the hole by the cutting.

The anchors 43a are fitted into the concave regions 41c and the base plate 43 is fixed to the tip of the tibia 41 via the adhesive (step S8). It is to be noted that as means for fixing the base plate 43 to the attaching surface 41b of the tibia 41, the screwing fixing structure using the screws or the like may be used.

Next, the articular facet surface 44 made of a resin is inserted into a surface side of the base plate and fixed thereto (step S9). Although not shown, the articular facet surface and the bonding surface of the base plate are beforehand constituted to be fittable with each other. It is to be noted that a structure other than the fitting structure may be used and, for example, fixing by use of the adhesive may be performed.

In the present embodiment, the attaching surface 41b of the tibia 41 is processed by using the ultrasonically vibrated treating section 14a of the probe 14 of the ultrasonic treatment device 2, whereby a flat surface having remarkably less unevenness can more accurately be formed as compared with processing by a conventional treatment tool such as a bar ablator. Furthermore, when the attaching surface 41b is the accurately flat surface, positional accuracy of the concave regions 41c to the anchors 43a of the base plate 43 also increases.

In consequence, dimensional accuracy of the positioning of the base plate 43 increases, close contact properties between the attaching surface 41b and the bonding surface of the base plate 43 enhance, a clearance is harder to be generated than before, and possibilities of generation of friction can be decreased. Therefore, the fixing of the base plate 43 to the tibia 41 strengthens, looseness is hard to be generated, and longer use is enabled.

### Second Embodiment

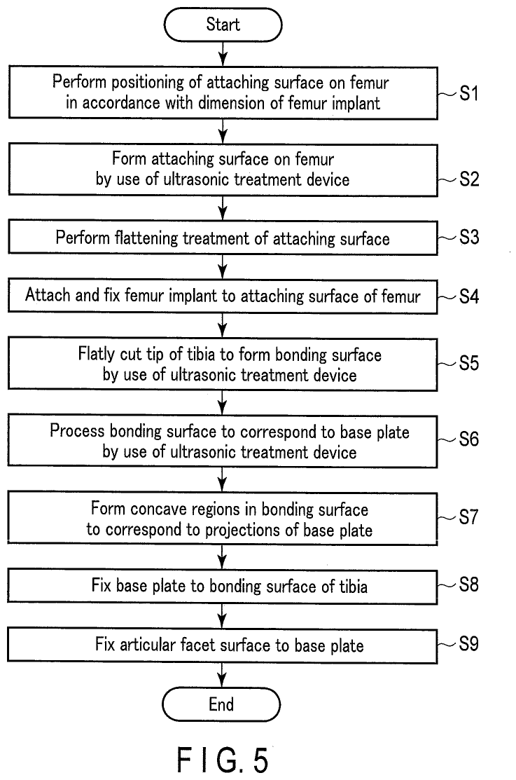

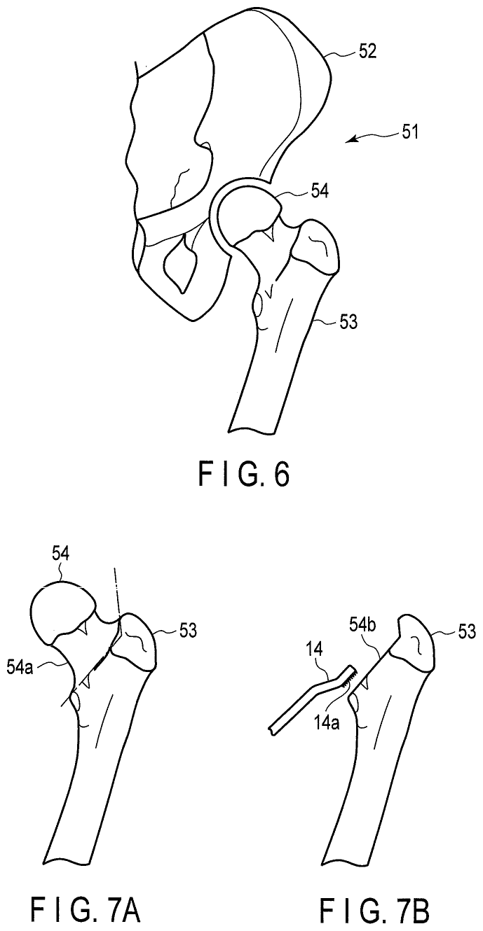

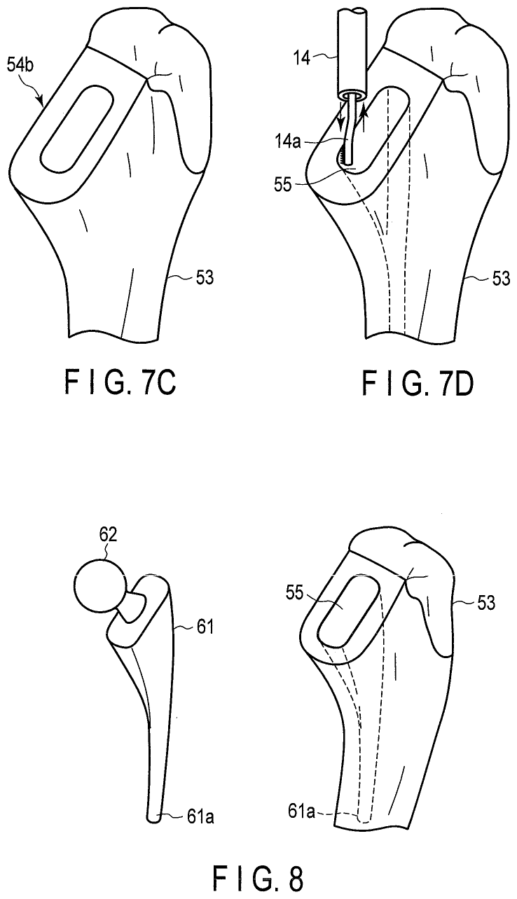

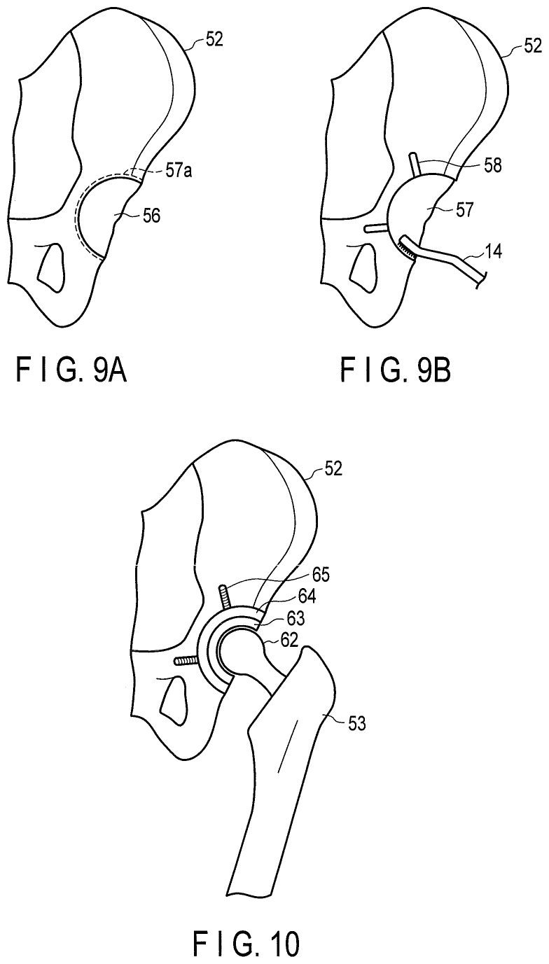

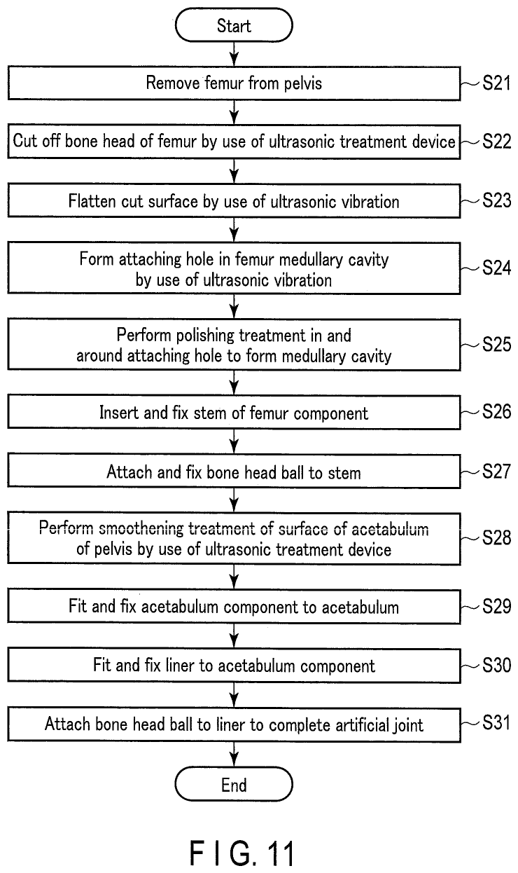

Next, with reference to surgical steps in FIG. 5, FIG. 6, FIG. 7A to FIG. 7D, and FIG. 8 to FIG. 11, there will be described a method for replacement arthroplasty of an artificial hip joint according to a second embodiment. FIG. 5 is a flowchart to explain surgical steps of the method for the replacement arthroplasty of the artificial hip joint. FIG. 6 is a view showing a pelvis and a femur which become targets of a replacement operation. FIG. 7A is a view to explain severance of the femur, FIG. 7B is a view showing a state of a flattening treatment of an attaching surface, FIG. 7C is a view showing the flattened attaching surface, and FIG. 7D is a view showing a state of forming a medullary cavity of the femur in a stem attaching hole by use of an ultrasonic treatment device. FIG. 8 is a view showing appearances of a femur component and the femur in which the medullary cavity is formed. FIG. 9A is a view showing an acetabulum of the pelvis which becomes a receiving port of the femur component, and FIG. 9B is a view showing a state of performing a flattening treatment of the acetabulum by use of the ultrasonic treatment device. FIG. 10 is a view showing a state where the femur component is attached to the pelvis. FIG. 11 is a view to explain surgical steps of the method for the replacement arthroplasty of the artificial hip joint.

FIG. 6 conceptually shows an original hip joint region 51 to which the artificial hip joint is attached. A clearance is generated between an acetabulum of a pelvis 52 and a bone head of a femur 53. First, in an excising step of the bone head, the femur is removed from the pelvis in a deformed joint region (step S21). Next, a position to cut off a bone head 54 of the femur 53 is set. In the present embodiment, as shown in FIG. 7A, a cutting line connecting a lesser trochanter to a greater trochanter is set in a boundary between a femur body and a femur neck 54a.

Afterward, under an environment in which the perfusion fluid circulates, the bone head 54 is excised along the set cutting line by use of an ultrasonic treatment device 2 (step S22). This excision can smoothly be performed with smaller force by use of ultrasonic vibration, handling is therefore easily performed, and furthermore, the excision along the intended line can easily be performed. Furthermore, as shown in FIG. 7B and FIG. 7C, unevenness of a cut surface 54b is eliminated and the surface is flattened with a treating section 14a of a probe 14 of the ultrasonic treatment device 2, so that the cut surface becomes a flat surface (step S23).

Next, as shown in FIG. 7D, under the environment in which the perfusion fluid circulates, an inner surface of a femur medullary cavity 55 is cut with the ultrasonically vibrated treating section 14a of the probe 14, and an attaching hole, into which a stem 61 is fittable, is formed (a reaming treatment) (step S24). Furthermore, a polishing treatment, i.e., a rasping treatment is performed in and around the formed attaching hole (step S25). The ultrasonic treatment device 2 is used in this treatment, whereby the surface can accurately be finished to obtain a noticeably smoother surface as compared with conventional surface roughness. Furthermore, the surface can be formed to match an appearance shape of the stem 61 and come in contact closely with the stem, and a clearance can be prevented from being generated between the stem 61 and the attaching hole.

Next, a tip 61a of the stem 61 that is the femur component is inserted into the attaching hole and fixed thereto (step S26). Furthermore, a bone head ball 62 is attached and fixed to a support base of the stem 61 (step S27).

Next, the treatment shifts to a treatment of an acetabulum 56 on a pelvis side which becomes a receiving port of the bone head ball 62. As shown in FIG. 9A, under the environment in which the perfusion fluid circulates, the flattening treatment to smoothen the surface of the acetabulum 56 is performed so that an acetabulum component 64 can be attached to the acetabulum 56 in which a defect on the pelvis side is generated, by use of the ultrasonic treatment device 2. Furthermore, as shown in FIG. 9B, screw holes 58 are formed and a receiving port 57 on the pelvis side is formed (step S28). Next, the acetabulum component 64 is fitted into the port and fixed thereto by using screws 65 (step S29), and a cup-shaped liner 63 of the artificial joint is fitted and fixed to the acetabulum component 64 (step S30).

As shown in FIG. 10, the bone head ball 62 fixed to the stem 61 is attached to the liner 63 on the pelvis side, thereby completing the artificial hip joint (step S31).

For the above-mentioned method for the replacement arthroplasty of the artificial hip joint of the present embodiment, the ultrasonic treatment device 2 is used in the treatment device for the formation of the femur medullary cavity 55 of the femur 53 shown in FIG. 7D, so that it is possible to obtain smooth surfaces as the medullary cavity inner surface and the cut surface 54a at accuracy noticeably higher than processing accuracy in a case where a conventional treatment tool is used. Therefore, the medullary cavity inner surface and the cut surface become the accurately smoothened surfaces, thereby increasing accuracy of an attaching dimension, and a clearance between the stem 61 and the medullary cavity inner surface is harder to be generated than before. As a result, there decrease possibilities of generation of friction due to rubbing of the stem 61 and the medullary cavity inner surface against each other, and it is possible to inhibit bone absorption in which a bone around the stem is melted.

Therefore, even when the artificial hip joint is attached and used for a long period of time, the generation of the clearance is inhibited, and looseness is hard to be generated.

In the formation of the acetabulum 57 shown in FIG. 9B, a smoothening treatment of the surface of the acetabulum 56 in which the defect is generated is performed by using the ultrasonic treatment device 2, and hence the acetabulum component 64 can more accurately be attached at a higher fitting degree of the component as compared with conventional processing. The treating section 14a of the distal end of the probe 14 may have a spherical surface or a ball shape as long as the surface of the acetabulum 57 is cut in the treatment. It is to be noted that, although not shown, small dimpled holes are suitably formed in the spherical surface or the surface of the ball shape.

According to the present embodiment, the smoothening treatment is performed by using the ultrasonic treatment device to increase the dimensional accuracy of the medullary cavity of the femur and the receiving port on the pelvis side (the acetabulum) and smoothen these surfaces, and hence the artificial joint can be attached to the pelvis and the femur without any clearances.

An ultrasound frequency of the ultrasonic treatment device in the present embodiment is transmitted at a frequency of, for example, 47 KHz, 23.5 KHz or the like. Additionally, an amplitude of the ultrasonic vibration is preferably from 50 μm to 200 μm. Furthermore, the ultrasound frequency and amplitude might be set to values other than the above values in accordance with design/specifications of the ultrasonic treatment device.

In the ultrasonic treatment device for use in the present embodiment, a treatment device main body containing the ultrasonic vibrator, the horn and the like is formed into a compact size that can easily be held in a hand. The treatment target region, e.g., the bone can be incised and cut by ultrasonically vibrating the distal end of the probe of the treatment device main body which is pressed against the bone.

The ultrasonic vibration is finely and continuously generated at a high rate, whereby the excised surface can be prepared in the form of a noticeably precisely excised surface having less unevenness as compared with a conventional excision technique.

Furthermore, the ultrasonic treatment device for use in the present embodiment quietly vibrates as compared with the conventional treatment tool, and hence the processing can exactly be performed and dimensional processing accuracy can be increased. Consequently, affinity for an implant improves, generation of wear debris due to the friction can be inhibited, and additionally, looseness of the implant itself can be inhibited, so that the artificial joint is usable longer than before.

Furthermore, in the bone excision in which the ultrasonic vibration is used, it is possible to cut the bone at noticeably high accuracy as compared with a drill, a shaver, a bar ablator or the like heretofore used in the method for the replacement arthroplasty. Additionally, the bone can not only be cut but also be shaved only by lightly attaching the ultrasonic treatment device against the bone, and hence the flattening or smoothening of the surface can easily be achieved. Therefore, the artificial joint can accurately be attached, generation of a worn material during the long use can be prevented, looseness can be inhibited, and longer use of the artificial joint is enabled.

Furthermore, there has been described the case where the cutting or surface polishing of the attaching surface to which the portion of the artificial joint is attached is performed by using the ultrasonic treatment device for the above-mentioned knee joint and hip joint, but the ultrasonic treatment device used for the artificial hip joint is easily applicable to a method for replacement arthroplasty of an artificial shoulder joint. For example, by using the ultrasonic treatment device, there is performed a reaming treatment or a rasping treatment of a receiving region in a humerus to attach an upper arm implant, or a reaming treatment or a rasping treatment to attach a glenoid to a scapula side.

### Third Embodiment

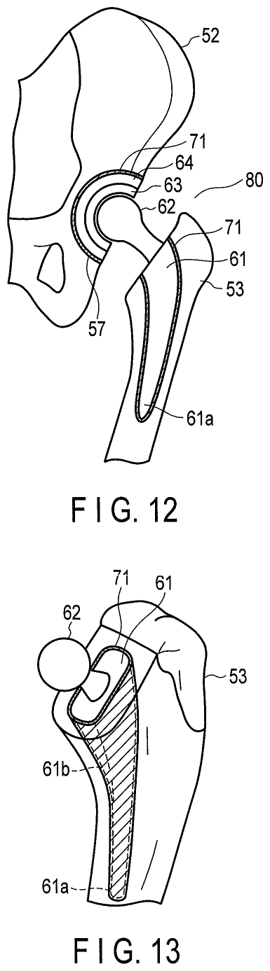

Next, with reference to FIG. 12 to FIG. 18, a revision operation of an artificial hip joint according to a third embodiment will be described. The present embodiment relates to a revision operation of an artificial joint which is used when an already used artificial joint, in which a defect occurs, is replaced with a new artificial joint. In this embodiment, an artificial hip joint is taken as an example of the artificial joint, and is described. This embodiment is applicable to a structure in which a component such as a stem is fixed to a bone by using cement.

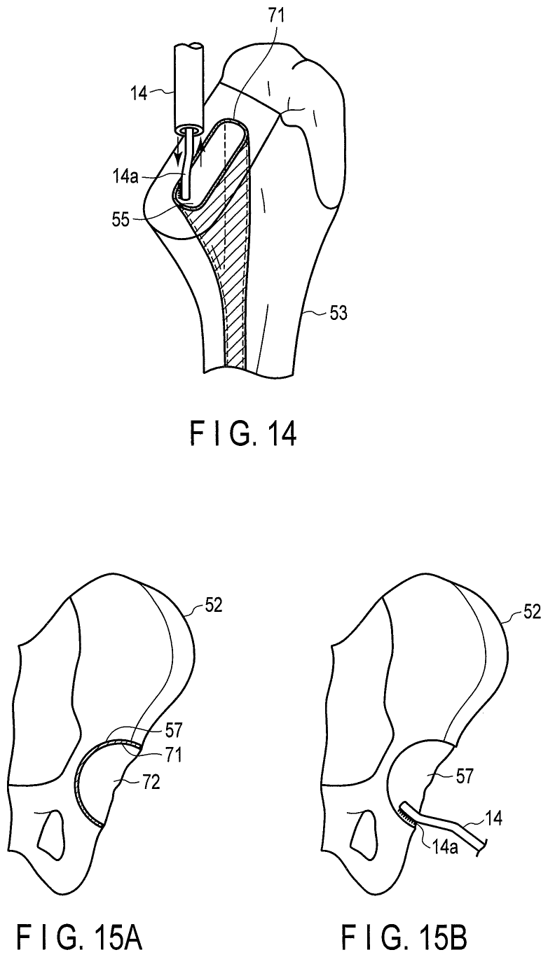

FIG. 12 is a view showing an artificial hip joint which is a target of a revision operation. FIG. 13 is a view showing a state of a femur which is a target of the revision operation. FIG. 14 is a view showing a state in which an attaching hole of a femur medullary cavity, from which a stem was removed, is treated by an ultrasonic treatment device. FIG. 15A is a view showing a state of an acetabulum at a time when an acetabulum component was removed from the pelvis. FIG. 15B is a view showing a state in which cement in the acetabulum and a remaining portion of the acetabulum component are removed by the ultrasonic treatment device, and a smoothing treatment is performed.

In an artificial hip joint 80 shown in FIG. 12, a femur component (stem 61) and an acetabulum component (cup) 64 are fixed to a femur 53 and an acetabulum 57, respectively, for example, by using cement 71 such as calcium phosphate-based bone cement. In the artificial hip joint 80, due to a gap resulting from a change with time, or wear by long-time use, or the like, looseness may occur in the stem 61 on the femur 53 side, or looseness may occur on a liner 63 on the acetabulum 57 side. In this case, when the artificial hip joint 80 cannot be repaired or when the artificial hip joint 80 is infected with germs, the artificial hip joint 80 needs to be changed for, that is, replaced with, a new artificial hip joint 80.

In the artificial hip joint 80, in the case where each component was fixed by using cement 71, it is difficult to remove the stem 61 and acetabulum component 64 at a time of replacing the artificial hip joint, and such a situation occurs that the cement 71 remains partly on an attachment location. Conventionally, the stem 61 and acetabulum component 64 are removed by using a cutting device such as a dedicated surgical tool or a rotary drill. However, in some cases, the cement 71 cannot completely be removed, and partly remains.

The cement 71 is harder than a bone portion in the femur medullary cavity. Even with use of the rotary drill or the like, the cement 71 is not easily removed. If an attempt is made to forcibly remove the cement, there is concern that a bone on a coalescence surface is removed together with the cement 71. Thus, it is difficult to flatten the surface of the attaching hole, as in the case of the time of first attaching the stem 61 and acetabulum component 64, and a rough surface with irregularities occurs. In addition, when each component is detached, such a situation may occur that a bone is unintendedly cut together with the cement, and the size of the attaching hole becomes larger than before the detachment. However, when infection occurs, it is necessary to remove a part of the infected bone together with the cement.

In the present embodiment, the ultrasonic treatment device used in the above-described first embodiment and second embodiment is used for the revision operation. The ultrasonic treatment device 2 removes the cement remaining on the attaching surface on which each component of the existing artificial hip joint was fixed, and the bone which is infected, and performs a process of re-flattening the attaching surface.

The revision operation of the present embodiment uses the surgical system 1 that is composed of the ultrasonic treatment device 2 shown in FIG. 2, the endoscope system 3, and the water-supply water-discharge section 5 that supplies and discharges the perfusion fluid such as saline. However, the endoscope system 3 is not indispensable, and the revision operation may be performed by the surgeon's direct sight.

A distal portion of the probe 14 includes a water-supply port 16c for supplying the perfusion fluid, and a water-discharge port 16d for sucking the perfusion fluid including removed broken pieces. In this revision operation, the cement removing treatment and the flattening treatment of the attachment location with use of the ultrasonic treatment device 2 are performed, in principle, in the environment in which the perfusion fluid is supplied and discharged and the perfusion fluid is circulated (filled). Needless to say, not in the environment in which the perfusion fluid flows, the treatments using the ultrasonic treatment device 2 can be performed. The probe 14 of the ultrasonic treatment device 2 used in the present embodiment is configured to include the treatment section 14a including at least one projection on a probe distal portion and on a part of a side surface which is continuous with the probe distal portion.

### Revision Operation of Femur Component

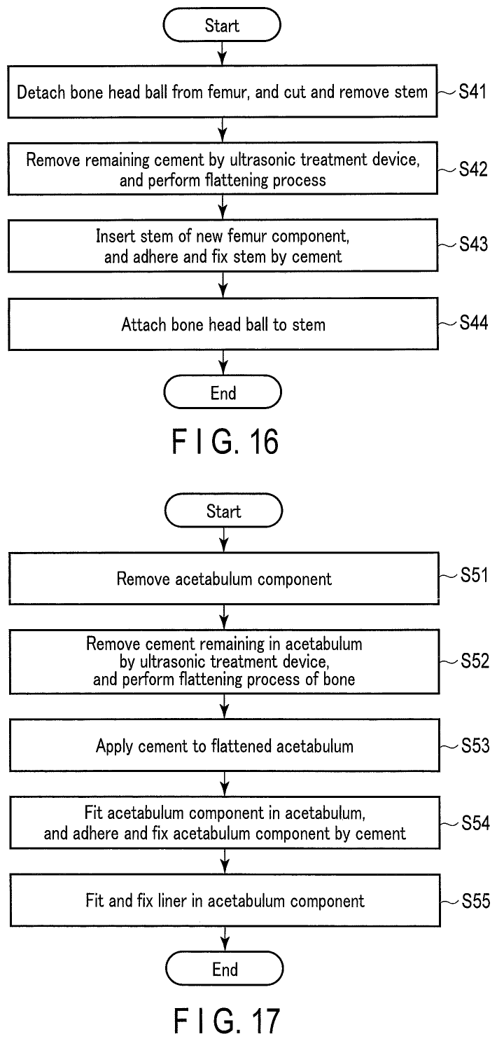

Next, referring to a flowchart of FIG. 16, a revision operation of a femur component in an artificial joint will be described.

FIG. 13 shows a state in which the stem 61 is fitted in the femur 53. The stem 61 is adhered and fixed by the cement 71 in the attaching hole formed in the femur medullary cavity of the femur 53. To begin with, after the bone head ball is removed, the stem 61 is detached from, or removed by cutting from, the femur 53 by using a dedicated tool, rotary drill or the like (step S41).

Next, as illustrated in FIG. 14, in the environment in which the perfusion fluid circulates (or an environment filled the perfusion fluid), the treating section 14a of the probe 14, which performs ultrasonic vibration, is abutted on the cement 71 which remains in the attaching hole of the femur 53, the cement 71 is broken and removed, and the attaching hole is regenerated (step S42). At this time, when the bone includes an infected portion, the infected bone portion is also removed. In the description below, as well as the cement, an infected bone, a stem 61 that is not removed and remains, and a portion of an acetabulum component to be described later, are targets of removal. Note that the remaining portion of the stem 61 is removed so as to be peeled off, by removing the cement 71 between the bone and the remaining portion of the stem 61.

In the removal of the cement or the like by the probe 14 which performs ultrasonic vibration, the projection portion of the treating section 14a (shown in FIG. 2) beats the cement (“hammering”), like a hammer, by ultrasonic vibration, and removes the cement by breaking the cement into fine pieces. At this time, since only the part that is in contact with the projection portion of the treating section 14a is removed, an unintended part is not removed. In addition, by making the probe 14, which has the projection at the distal end thereof, reach the bottom of the attaching hole, the cement remaining in the hole bottom can also be removed. Furthermore, a polishing process by the treating section 14a of the probe 14 that performs ultrasonic vibration is applied to the surface of the attaching hole from which the cement 71 has been removed. Thereby, the surface of the bone can be flattened.

A new cement 71 is applied to the surface of the attaching hole of the femur 53, which has been regenerated by the flattening process. Then, a stem 61 of a new femur component is inserted in the attaching hole, and fixed by adhesion with the cement 71 (step S43). At the time of the fixing, the new cement 71 may be applied to the surface of the attaching hole, or may be applied to the stem 61 of the new femur component. Thereafter, the bone head ball 62 is attached to the stem 61 (step S44), and the revision in the femur component is completed.

### Revision Operation of Acetabulum Component

Next, referring to a flowchart of FIG. 17, a revision operation of an acetabulum component in the artificial joint will be described.

To begin with, after the femur component (bone head ball) is detached from the artificial joint 80 shown in FIG. 12, the liner 63 is removed. Further, the acetabulum component (cup) 64 is detached from, or removed by cutting from, the acetabulum 57 by using a dedicated tool, rotary drill or the like (step S51). FIG. 15A shows the acetabulum 72 in the state in which the cement 71 remains after the detachment.

Further, as illustrated in FIG. 15A and FIG. 15B, in the environment in which the perfusion fluid circulates, the treating section 14a of the probe 14, which performs ultrasonic vibration, is abutted on the cement 71 which remains in the acetabulum 57, and the cement 71 is broken and removed. Further, as illustrated in FIG. 15B, like the attaching hole of the femur, the inner curved surface of the acetabulum 57 is subjected to the flattening process by using the treating section 14a so that the inner curved surface may become smooth with no irregularities, and the acetabulum 57 is regenerated (step S52). In addition, as described above, when the bone includes an infected portion, the infected bone portion is also removed. Note that when there remains a target of removal such as a part of the acetabulum component, the target of removal is also removed.

Next, a new cement 71 is applied to the acetabulum 57 that has been regenerated by the flattening process (step S53). A new acetabulum component 64 is fitted in, and adhered and fixed by the cement 71 (step S54). Thereafter, the cut-shaped liner 63 is fitted and fixed in the acetabulum component 64 (step S55), and the revision operation of the acetabulum component is completed.

### Revision Operation of Femur Component and Acetabulum Component

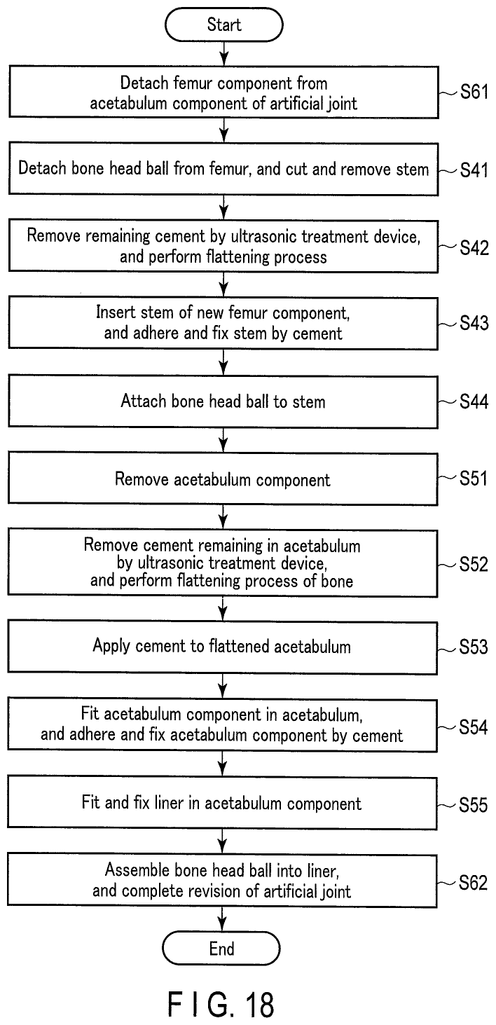

Hereinafter, referring to a flowchart of FIG. 18, a description will be given of a revision operation of successively replacing both the femur component and acetabulum component of the artificial joint. In the description below, surgical steps, which are the same as in the revision operations of the femur component and acetabulum component in the above-described FIG. 16 and FIG. 17, are denoted by the same step numbers, and a detailed description thereof is omitted. In the example to be described below, the revision operation of the femur component is first performed, and then the revision operation of the acetabulum component is performed. Needless to say, the revision operation of the acetabulum component may first be performed, and then the revision operation of the femur component may be performed.

To start with, the femur component side is detached from the acetabulum component of the artificial hip joint 80 shown in FIG. 12 (step S61). Specifically, the bone head ball 62 is detached from the acetabulum component. In addition, if much time is needed for detaching the bone head ball 62, a support portion between the bone head ball 62 and the stem 61 may be cut off.

Next, the stem 61, which is fixed in the attaching hole with use of the cement 71, is detached from, or removed by cutting from, the femur 53 by using a dedicated tool, rotary drill or the like (step S41).

Next, as illustrated in FIG. 14, in the environment in which the perfusion fluid circulates, the treating section 14a of the probe 14, which performs ultrasonic vibration, is butted on the cement 71 which remains in the attaching hole of the femur 53. By the ultrasonic vibration of the treating section 14a, the cement 71 is broken and removed, the flattening process of the bone is performed, and the attaching hole is regenerated (step S42). At this time, when the bone includes an infected portion, the infected bone portion is also removed.

After a new cement 71 is applied to the surface of the attaching hole which has been regenerated, a stem 61 of a new femur component is inserted in the attaching hole, and fixed by adhesion with the cement 71 (step S43). Thereafter, the bone head ball 62 is attached to the stem 61 (step S44).

Subsequently, the revision operation of the acetabulum component 64 on the pelvis side is performed. To begin with, the liner 63 is removed from the acetabulum component 64. Further, the acetabulum component 64 is detached from, or removed by cutting from, the acetabulum 57 by using a dedicated tool, rotary drill or the like (step S51).

Next, in the environment in which the perfusion fluid circulates, the treating section 14a of the probe 14, which performs ultrasonic vibration, is abutted on the cement 71 which remains in the acetabulum 57, and the cement 71 is broken and removed. Further, the inner surface of the acetabulum 57 is flattened, and the acetabulum 57 is regenerated (step S52). At this time, if the bone includes an infected portion, the infected bone portion is also removed.

Next, a new cement 71 is applied to the regenerated acetabulum 57 (step S53). A new acetabulum component 64 is fitted in, and adhered and fixed by the cement 71 (step S54). Thereafter, the liner 63 is fitted and fixed in the acetabulum component 64 (step S55).

Further, in the same manner as illustrated in FIG. 12, the bone head ball 62 fixed to the stem 61 is assembled in, and coupled to, the liner 63 on the pelvis side, and the revision of the artificial hip joint is completed (step S62).

According to the above-described present embodiment, it is possible to completely remove the cement remaining in the attaching hole and the acetabulum, after the femur component (stem 61) and acetabulum component 64 are detached for revision, and to flatten the surfaces of the attaching hole and acetabulum by eliminating irregularities on the surfaces.

The cement, which is not easily removed by the conventional surgical tool, rotary drill or the like, is finely broken and removed by ultrasonic vibration. Thus, even when cement remains in a mottled manner after each component is detached, the cement can completely be removed. In addition, in a revision operation in which the cement is partly left, a desired amount of cement can be left on a desired location. Therefore, the components can be attached as planned.

In the case of the ultrasonic treatment device, only a part on which the treating section 14a of the probe 14 is abutted is removed, and there occurs neither scattering of a part that is put in contact with a hard portion of a rotary drill or the like, nor cutting of an entire part around a drill blade. Thus, the surgeon can remove a desired part by a desired amount. Moreover, since there occurs no scattering as in the case of the rotary drill, no damage is caused on an unintended part by contact. Accordingly, the size of the attaching hole at the time of attaching the stem or acetabulum component does not increase due to excessive cutting of the bone. Besides, since the ultrasonic treatment device can form a flat cut surface, the femur stem 61 and acetabulum component 64 for the revision can be fitted, without a gap, in the respective regenerated attaching holes.

Furthermore, since the ultrasonic treatment is performed in the environment in which the perfusion fluid circulates, a temperature rise of the probe 14 is suppressed, and a thermal influence on the bone part that is the target of treatment can be reduced. In addition, since pieces of cement or bone dust, which is pulverized by the probe 14 that performs ultrasonic vibration, is taken out from the removal location together with the perfusion fluid, the latest condition of the removal surface can be confirmed. Therefore, the removal state of the cement can be exactly understood, and excessive cutting of bones can be prevented.

Besides, in the case of conventional surgical tools including a rotary drill and the like, the removal of the cement and the removal of the bone cannot be performed by the same tool, and treatments need to be performed by changing tools in accordance with purposes of use. By contrast, in the case of the ultrasonic treatment device, the cement and bone can be removed without changing tools. In addition, an infected bone can be removed together with the cement. Further, since the ultrasonic treatment is performed in the environment of the perfusion fluid, when the infected bone is removed, bone dust including the infected bone is discharged from the body of the patient such that the bone dust is washed away along with the perfusion fluid, and infection after the revision operation can be prevented.

The above-mentioned present embodiment also includes the following gist.

(1) There is provided an ultrasonic method for replacement arthroplasty which is performed by using a system comprising an ultrasonic transmitter (a power source device), an ultrasonic vibrator and an ultrasonic probe, further comprising water supply means and being capable of supplying water from a probe distal end. In consequence, when bone excision/formation is performed by using ultrasonic vibration, effects of heat which could be generated can be decreased.

(2) There is provided an ultrasonic method for replacement arthroplasty which comprises an ultrasonic transmitter (a power source device), an ultrasonic vibrator and an ultrasonic probe, and in which at a time of formation of a medullary cavity, bone excision/formation is performed under water while filling the medullary cavity with the water (perfusion fluid). In consequence, when the bone excision/formation is performed by using ultrasonic vibration, generated heat effects can be decreased.

(3) There is provided an ultrasonic method for replacement arthroplasty which comprises an ultrasonic transmitter (a power source device), an ultrasonic vibrator, an ultrasonic probe and an arthroscopic system, and in which at a time of formation of a medullary cavity, bone excision/formation is performed under water while confirming an excised region with an arthroscope. When the bone excision/formation is performed by using ultrasonic vibration, it is possible to decrease heat effects which could be generated and to more exactly and securely perform the excision.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

1. A method for revision arthroplasty, comprising:
detaching an existing femur component of an artificial joint from a femur, the existing femur component being fixed to the femur by cement, and the artificial joint being composed of the existing femur component and an acetabulum component;
breaking and removing the cement and bone remaining in an attaching of the femur from which the existing femur component was detached, by transmitting ultrasonic vibration to a treating section provided at a probe distal end of an ultrasonic treatment device when the treating section is brought into contact with the cement and the bone, and regenerating an attaching hole of the femur for a new femur component;
applying cement in the regenerated attaching hole of the femur; and
attaching and fixing the new femur component in the regenerated attaching hole of the femur.

2. The method of claim 1, further comprising:
assembling and coupling the new femur component and the acetabulum component.

3. The method of claim 1, wherein the breaking and the removing of the cement and the bone includes flattening a surface of the attaching hole of the femur by the transmitting of the ultrasonic vibration.

4. The method of claim 1, wherein a process of the breaking and the removing by the treating section of the ultrasonic treatment device in the attaching hole of the femur is performed in an environment filled the perfusion fluid.

5. The method of claim 1, wherein the bone remaining in the attaching of the femur includes a bone infected with germs.

6. A method for revision arthroplasty, comprising:
detaching an existing acetabulum component of an artificial joint from an acetabulum, the existing acetabulum component being fixed to the acetabulum by cement, and the artificial joint being composed of the existing acetabulum component and a femur component;
breaking and removing the cement and bone remaining in the acetabulum from which the existing acetabulum component was detached, by transmitting ultrasonic vibration to a treating section provided at a probe distal end of an ultrasonic treatment device when the treating section is brought into contact with the cement and the bone, and regenerating the acetabulum for a new acetabulum component;
applying cement to the regenerated acetabulum; and
attaching and fixing the new acetabulum component in the regenerated acetabulum.

7. The method of claim 6, further comprising:
assembling and coupling the femur component and the new acetabulum component.

8. The method of claim 6, wherein the breaking and the removing of the cement and the bone includes flattening a surface in the acetabulum by the transmitting of the ultrasonic vibration.

9. The method of claim 6, wherein a process of the breaking and the removing by the treating section of the ultrasonic treatment device in the acetabulum is performed in an environment filled the perfusion fluid.

10. The method of claim 6, wherein the bone remaining in the acetabulum includes a bone infected with germs.

11. A method for revision arthroplasty, comprising:
detaching, from an artificial joint composed of an existing femur component and existing acetabulum component fixed by cement, the femur component;
a femur component revision arthroplasty including:
detaching the existing femur component of the artificial joint from a femur;
breaking and removing the cement and bone remaining in an attaching of the femur from which the existing femur component was detached, by transmitting ultrasonic vibration to a treating section provided at a probe distal end of an ultrasonic treatment device when the treating section is brought into contact with the cement and the bone, and regenerating an attaching hole of the femur for a new femur component; and
applying cement in the regenerated attaching hole of the femur; and
attaching and fixing the new femur component in the regenerated attaching hole of the femur; and
an acetabulum component revision arthroplasty including:
detaching the existing acetabulum component of the artificial joint from an acetabulum,
breaking and removing the cement and the bone remaining in the acetabulum from which the existing acetabulum component was detached, by the transmitting of the ultrasonic vibration to the treating section when the treating section is brought into contact with the cement and the bone, and regenerating the acetabulum for a new acetabulum component;
applying cement to the regenerated acetabulum; and
attaching and fixing the new acetabulum component in the regenerated acetabulum,
wherein the femur component revision arthroplasty and the acetabulum component revision arthroplasty are performed in any order; and
assembling and coupling the new femur component and the new acetabulum component.
